(19) United States
(12) Patent Application Publication
(10) Pub. No.: US 2018/0071424 A1
(43) Pub. Date: Mar. 15, 2018

(54) SCENT DISPERSING APPARATUS WITH DECORATIVE PLANT FEATURE

(71) Applicant: Ntxhuav Kong, Nixa, MO (US)

(72) Inventor: Ntxhuav Kong, Nixa, MO (US)

(21) Appl. No.: 15/266,585

(22) Filed: Sep. 15, 2016

(51) Int. Cl. A61L 9/03

(52) U.S. Cl. CPC A61L 9/03; A61L 2209/133

(57) ABSTRACT

A scent dispersing apparatus is disclosed. The apparatus comprises a shell that extends upwardly from a base and a receptacle for holding a scent producing substance. In one embodiment the receptacle rests on a ledge extending inwardly from a sidewall of the shell. Atop the receptacle is a plant feature that has a synthetic or organic plant extending upwardly. The invention may include a spacer ring inserted between the plant feature and receptacle. The apparatus includes a heat source for melting the scent producing substance. Scent given off by the scent producing substance could be made to match the scent given off by the plant in the plant feature if the plant was found in nature.

### FIELD

The present invention relates generally to scent dispersing devices. More specifically, the invention relates to such devices having a decorative plant feature and that operate by heating a scent producing substance.

### BACKGROUND

Various types of devices exist for dispersing scent into a space. The most basic type of device is a scented candle that releases fragrance when burned. Another type of device is a perfume or scented oil diffuser that allows the scented oil to vaporize off of saturated reeds. Electrically powered devices have become popular as the fragrance given off can be adjusted to give off a fragrance of uniform strength for a certain amount of time. Such electrical devices include air fresheners mounted to electrical receptacles and table mounted wax warmers. Wax warmers have become popular because of their aesthetically pleasing design combined with their low maintenance operation.

A typical wax warmer comprises a ceramic base which houses a heating element and a removable dish for holding wax cubes. The removable dish is placed atop the base and the wax is directly over the heating element. As the wax melts, a scent is released similar to how scent is released from a burning scented candle. The liquefied wax is typically allowed to cool and solidify before cleaning the warmer. Cleanup is simple because the hardened pool of wax can be easily scraped from the removable dish prior to the next use. An appealing aspect of wax warmers is that the scent can easily be changed by selecting wax cubes with different scents. Also, wax warmers typically have a long operational life with little maintenance. Aside from cleaning and replacing wax, the warmers typically only require occasional replacement of the heating element, which may take the form of an incandescent light bulb. The low maintenance, ease of operation, and customizable scents are features that have made wax warmers a popular scent dispersing device.

Others have attempted to create air fresheners in combination with a plant or floral feature. For example, U. S. Patent Application Pub. No. 2009/0280038 describes a floral arrangement in combination with an air freshener. Similarly, Chinese Patent No. CN201033174Y and Japanese Patent No. JPH0650566U disclose flower pots having fragrance diffusers. However, none of these devices combine a plant feature with the convenience and effectiveness of a wax warmer.

Thus, there remains a need in the art for a scent dispersing device that has the benefits of a wax warmer combined with the aesthetic appeal of a floral or plant accent.

### SUMMARY

The present invention is directed to a scent dispersing apparatus comprising a shell that extends upwardly from a base. The shell defines an interior void and may have one or more ventilation openings formed in a sidewall of the shell to allow scent and light to project through the openings. The shell may also include a ledge that extends from the shell sidewall into the interior void. Preferably, but not necessarily, the ventilation openings are located below the ledge.

The invention further comprises a receptacle for holding a scent producing substance and the receptacle may have a sidewall with one or more ventilation openings formed therein. A portion of the receptacle may be positioned atop the ledge. Also included in the invention is a plant feature comprising a synthetic or organic plant extending upwardly from a mount. The plant feature is supported by the receptacle, and there may be a spacer ring inserted between the plant feature and receptacle. The mount may have a channel into which a portion of the receptacle is inserted, or the spacer ring may have such a channel.

Additionally, an electric heat source for melting the scent producing substance may be located in the interior void within the shell, below the receptacle. The heat source may also give off light.

### BRIEF DESCRIPTION OF THE DRAWINGS

FIG. 1 is a perspective view of a scent dispersing apparatus with plant feature in accordance with a first exemplary embodiment of the present invention.

FIG. 2 is an exploded view of the scent dispersing apparatus with plant feature of FIG. 1.

FIG. 3 is a cross sectional view of the scent dispersing apparatus with plant feature of FIG. 1.

FIG. 4 is an exploded view of a scent dispersing apparatus with plant feature in accordance with a second exemplary embodiment of the present invention.

FIG. 5 is a cross sectional view of a warming receptacle with spacer ring and a plant feature mounted thereon.

FIG. 6 includes exploded views of a plant feature comprising lilies and a warming receptacle containing lily-scented wax, and a plant feature comprising roses and a warming receptacle containing rose-scented wax.

### DETAILED DESCRIPTION OF EXEMPLARY EMBODIMENTS

Various embodiments of the present invention are disclosed herein, it is to be understood that the disclosed embodiments are merely exemplary of the invention, which may be embodied in various forms. Thus, any specific structural and functional details disclosed herein are not to be interpreted as limiting, but merely as a basis for the claims and as a representative basis for teaching one skilled in the art to variously employ the present invention in virtually any appropriately detailed structure. The drawings constitute a part of this specification and include exemplary embodiments of the present invention and illustrate various objects and features thereof.

Certain terminology used in the following description is for convenience in reference only and is not limiting. For example, the words “vertically,” “horizontally,” “vertical,” “horizontal” and “upwardly,” “downwardly,” “upper,” “lower” all refer to the depicted position of the element or embodiment to which the reference is made. The words “inwardly” and “outwardly” refer to directions toward and away from, respectively, the geometric center of the embodiment being designated and parts thereof. The terminology used herein may include the words specifically mentioned, derivatives thereof and words of a similar import. It is further understood that terminology such as the aforementioned directional phrases may be used to describe exemplary embodiments of the scent dispersing apparatus as shown in the figures herein. This is for convenience only as it is understood that the exemplary embodiments of the apparatus described may also be used in other orientations.

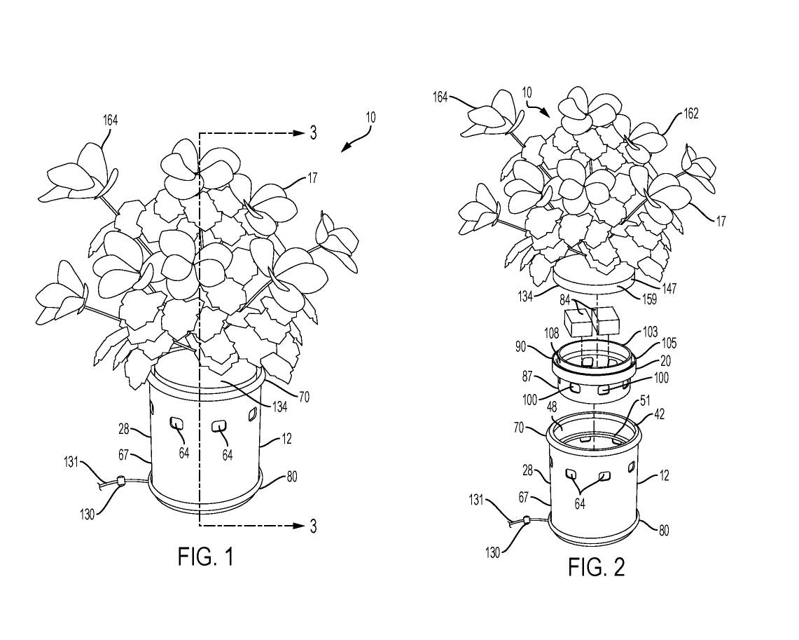

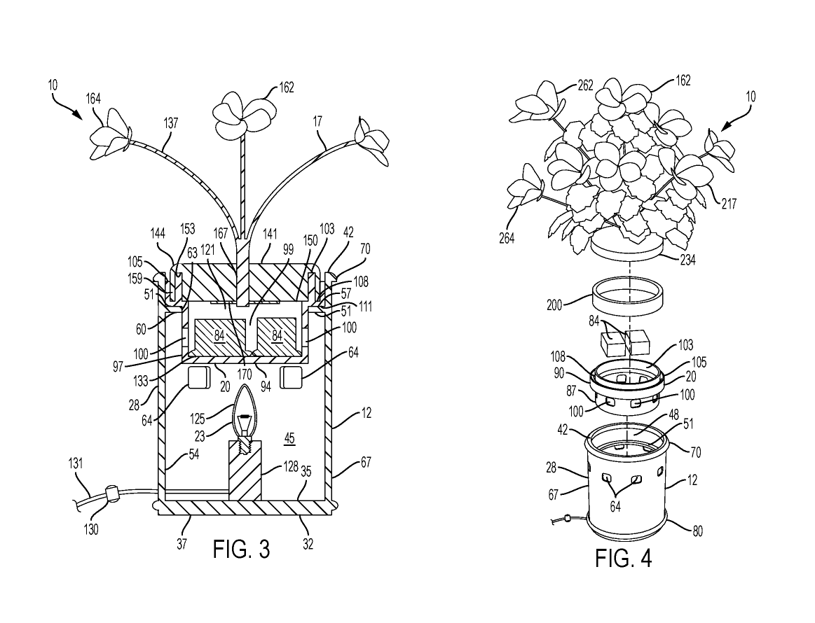

Looking first to FIG. 1, a scent dispersing apparatus with decorative floral or plant feature in accordance with an exemplary embodiment of the present invention is depicted generally by the numeral 10. The scent dispersing apparatus 10 comprises a housing 12, a decorative plant feature 17, and a warming receptacle 20. As shown in FIGS. 2 and 3, plant feature 17 sits atop warming receptacle 20 which rests inside the upper end of housing 12. Mounted inside housing 12 is a heat source 23. It should be understood that while the scent dispersing device of FIG. 1 is shown having a decorative plant feature utilizing flowers, the present invention anticipates and encompasses other types of plant themed decorative features (both organic and synthetic) such as cacti, succulents, plants, greenery, miniature trees and shrubs. It should be further understood that the embodiments described herein are directed to a cylindrical scent dispersing device, but the invention may come in other shapes such as cuboid, pyramidal, and frustoconical (including inverted variations of each).

Looking to FIG. 2, housing 12 comprises a vertically oriented cylindrical shell 28 extending upwardly from a circular base 32. Base 32 takes the general form of a disc with a substantially planar upper surface 35 and a substantially planar lower surface 37; the lower surface 37 may include feet or a downwardly extending protrusion (not shown) for elevating base 32 slightly above the surface on which it is located. Shell 28 extends upwardly from the perimeter of base 32 and terminates in a rim 42 encircling upper opening 48. Shell 28 surrounds an internal void 45.

Shell 28 includes an annular ledge 51 attached to the interior surface 54 of shell 28. As shown in FIG. 3, ledge 51 protrudes away from the interior surface 54 into void 45.

The ledge 51 has an upper surface 57, a lower surface 60, and an inwardly facing surface 63 configured such that ledge 51 has a rectangular cross section. Ledge 51 preferably forms a continuous unbroken ring around the interior of shell 28 but it is foreseen that the ledge 51 could comprise one or more evenly spaced segments protruding from interior surface 54.

Housing 12 further includes one or more ventilation openings or vents 64 formed in shell 28 below ledge 51. The vents 64 are evenly spaced circumferentially around shell 28 and are preferably located on the upper half of the shell. Vents 64 are sized and shaped to allow sufficient airflow to carry a scent from void 45 to the area outside of shell 28. The vents 64 also allow light to shine from void 45 through the vents. The light shining through the vents 64 may be used for decorative purposes or the light may be used to illuminate poorly lit areas. The size, shape and quantity of vents 64 will depend on the specific application and use of apparatus 10; however a typical embodiment may consist of eight half-inch square vents as shown in FIG. 2.

Housing 12 further includes annular protrusions on the exterior surface 67 of shell 28 to increase the aesthetics of apparatus 10 and make the apparatus easier to grip. One such protrusion is overhang 70 located proximate rim 42. Overhang 70 includes a downwardly extending arcuate top 73 and a planar underside 75. Another such annular protrusion is ridge 80 proximate base 32.

As shown in FIG. 2, apparatus 10 further comprises a warming receptacle 20 that holds pieces of meltable scent producing substance 84 and is receivable in upper opening 48. FIGS. 2 and 3 illustrate that warming receptacle 20 is generally cylindrical and has a multi-tier shape. The receptacle 20 includes a lower tier 87 and an upper tier 90. Lower tier 87 has a circular bottom wall 94 and a cylindrical side wall 97 extending upwardly from the bottom wall 94. Side wall 97 encircles a lower tier void 99. Interior ventilation openings or vents 100 are formed in side wall 97 to allow a scent to move from void 99 to an area outside of warming receptacle 20. The size, shape, location and quantity of vents 100 will depend on the specific application and use of apparatus 10; however, as shown in FIG. 2, the vents 100 are typically evenly spaced circumferentially around side wall 97 and are sized and shaped similar to the vents 64 formed in shell 28.

As best seen in FIGS. 2 and 3, the upper tier 90 of receptacle 20 is located directly above lower tier 87. Upper tier 90 comprises concentric cylindrical sidewalls 103 (inner) and 105 (outer) with a channel 108 formed therebetween. Outer sidewall 105 extends upwardly from an outer overhang 111, which projects outwardly from sidewall 97. Overhang 111 connects to side wall 97 at a circumferential corner 115. Overhang 111 projects from sidewall 97 for a distance that is approximately the same as the width of ledge 51. The underside 118 of overhang 111 is generally planar and rests on the upper surface 57 of ledge 51 when the warming receptacle 20 is positioned in housing 12.

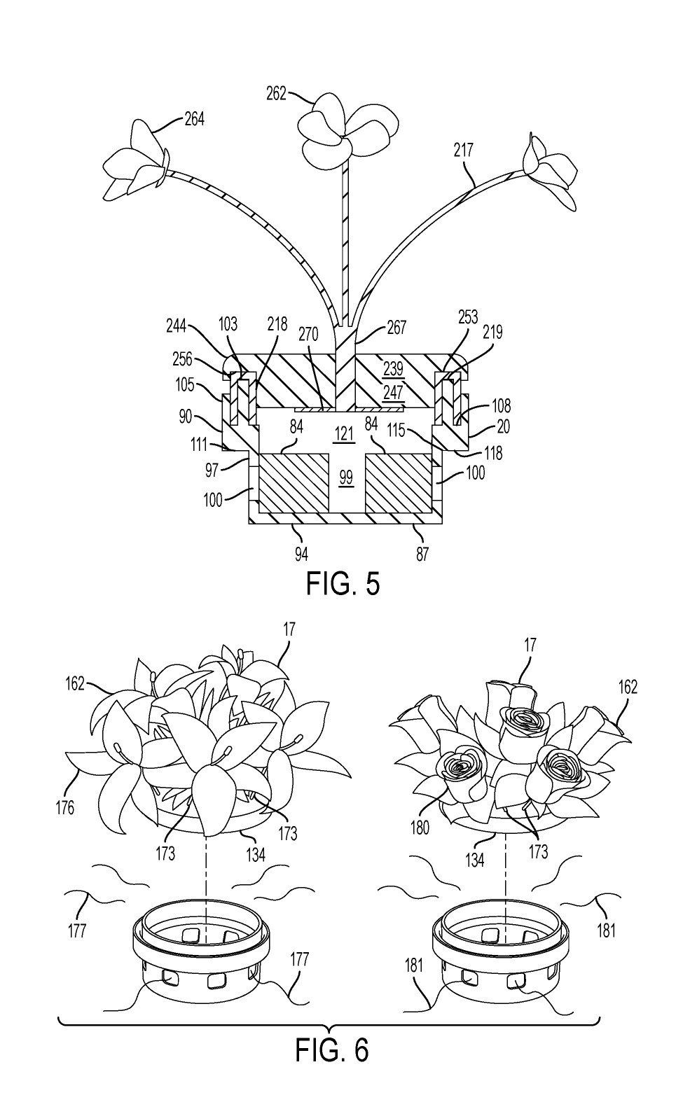

Inner sidewall 103 of upper tier 90 surrounds an upper tier void 121. Upper tier void 121 is in communication with lower tier void 99 and both voids have a similar circular shape. FIGS. 3 and 5 best illustrate that inner sidewall 103 is spaced inward from outer sidewall 105 to form channel 108. The inner sidewall 103 is generally parallel to outer sidewall 105 and is slightly taller than outer sidewall 105. Inner sidewall 103 may be located directly above lower tier side wall 97 to form a continuous interior wall surface from the bottom of lower tier 87 to the top of upper tier 90, or it may be spaced outwardly from wall 97 as shown in FIG. 3. The exact height and position of inner sidewall 103 will depend on the size of the plant feature 17 that sits atop warming receptacle 20. In a typical configuration, sidewall 103 will be three quarters of an inch tall and spaced outwardly from the lower tier side wall 97 by approximately one quarter of an inch.

As shown in FIG. 3, heat source 23 is mounted inside housing 12. The heat source 23 is located within void 45 and is typically located on supper surface 35 of base 32. The heat source 23 may or may not be secured to upper surface 35. It is foreseen that the heat source 23 may be mounted in other locations within the housing 12 such as interior surface 54 of shell 28 so long as the heat given off by the heat source is able to be distributed across the bottom wall 94 of warming receptacle 20. Heat source 23 may even be mounted to warming receptacle 20. Heat source 23 may also give off light, and the light may shine through vents 64 for decorative purposes or to illuminate poorly lit areas. A preferred type of heat source is an incandescent light bulb 125 with the socket 128 being secured proximate the center of upper surface 35; however other types of heat sources may be used. The bulb 125 is preferably located close enough to the warming receptacle 20 such that the heat will permeate the bottom wall 94 of the receptacle and melt the contents of the receptacle. The bulb 125 is controlled by a switch 130 mounted outside housing 12 on the power cord 131 serving the bulb. The power cord 131 plugs into a conventional 120 volt receptacle (not shown). It is foreseen that heat source 23 may be electrically powered by other means including a battery.

A scent producing substance 84 is placed on bottom wall 94 of warming receptacle 20 in the lower tier void 99. The scent producing substance 84 may be of any scented substance known in the art, but the preferred substance is believed to be scented petroleum based wax. The substance is typically sold in cubes or tarts which can be placed in warming receptacle 20. The number of cubes used in apparatus 10 will depend on how strong the scent produced by the apparatus is intended to be. The more cubes of scent producing substance 84 placed in warming receptacle 20, the stronger the scent produced by apparatus 10 will be. While the cubes of substance 84 may have a slight scent when solid, the scent is generally stronger when the substance 84 is melted. Heat source 23 is generally hot enough, and located close enough to warming receptacle 20, to melt the pieces of scent producing substance 84 into a scent dispersing liquid 133.

Scent dispersing apparatus 10 further comprises a decorative floral or plant feature 17 that sits atop warming receptacle 20. Plant feature 17 includes a mount 134 and plant accent 137. Mount 134 has a horizontal upper surface 141 from which the plant accent 137 projects. Upper surface 141 is typically circular and may have a radiused corner 144. Extending downward from the radius corner 144 is the circumferential face or vertical surface 147 of mount 134. Surface 147 extends downward to a horizontal lower surface 150 that approximates the size and shape of upper surface 141. A channel or groove 153 is formed in lower surface 150 proximate its periphery 156. The portion of mount 134 between groove 153 and vertical surface 147 is sidewall 159. The depth of groove 153 approximates the height of inner sidewall 103 of warming receptacle 20, and the width and radius of curvature of groove 153 approximates the width and radius of curvature of inner sidewall 103. When plant feature 17 is placed on warming receptacle 20, inner sidewall 103 should be received completely within groove 153 and sidewall 159 should be received within channel 108 of warming receptacle 20.

Plant accent 137 includes a plant 162. As shown in the drawings, the present embodiment comprises a plant 162 having synthetic flowers 164; however as noted above the plant may comprise other types of plant themed objects (both synthetic and organic). The flowers 164 include stems connected to a central trunk 167. As shown in FIG. 3, trunk 167 passes through mount 134 and is held therein by a retaining member 170 attached to lower surface 150. Retaining member 170 may be any device commonly known in the art such as staples, adhesive, tacks or any other method of retaining synthetic floral stems in a piece of material. It is foreseen that mount 134 could be made of foam or other type of easily penetrated material and a retaining member 170 may not be necessary because the trunk 167 can be pushed into position without completely penetrating mount 134. FIG. 6 shows that, alternatively, flowers can be individually mounted in the mount 134 with each stem 173 attaching to the mount 134 rather than the flower stems being combined to a central trunk 167 that attaches to the mount 134.

As shown in FIG. 6, the type of scent given off by the scent producing substance 84 may depend on the design of the plant accent 137. It may be desired that the scent produced by the apparatus be paired with the plant accent 137. For example, if plant accent 137 comprises lilies 176, the apparatus 10 may produce a lily scent 177. Or if plant accent 137 comprises roses 180, the apparatus 10 may produce a rose scent 181. Moreover, scents may be tied to seasons or holidays. At Christmas a pine or cinnamon scent may be used, whereas at Thanksgiving a pumpkin scent may be used.

An alternate embodiment of the present invention is shown in FIGS. 4 and 5. The alternate embodiment includes many of the same components as the previously described embodiment except that a spacer ring 200 (also referred to herein as simply a “spacer”) is placed between the warming receptacle and plant accent. The spacer ring 200 can be used with warming receptacle 20 however an alternate plant feature 217 must be used. One benefit to using spacer ring 200 is to elevate the plant feature 217 above warming receptacle 20 and housing 12 so that it is more visible to an individual. Another benefit is that spacer ring 200 makes it easier to correctly position the plant feature 217 with respect to warming receptacle 20 and ensure that the plant feature is properly engaged with and secured in place by the warming receptacle 20.

Spacer ring 200 comprises an annular member 218 having an inverted U-shaped cross section and a channel 219. The depth of channel 219 approximates the height of inner sidewall 103 of warming receptacle 20, and the width and radius of curvature of channel 219 approximates the width and radius of curvature of inner sidewall 103. When spacer ring 200 is used, it is placed on warming receptacle 20 such that inner sidewall 103 is received within channel 219 and then plant feature 217 is placed on the spacer ring 200. Spacer ring is typically annular, but non-annular spacers are also foreseen. As an example, spacers could be positioned radially around the warming receptacle 20 rather than having a continuous spacer ring.

Plant feature 217 includes a mount 234 and plant accent 237. Mount 234 has an upper portion 239 with a horizontal upper surface 241 from which the plant accent 237 projects. Upper surface 241 is typically circular and may have a radiused edge 244. Mount 234 also has a circular lower portion 247 that is smaller in diameter than upper surface 241; the diameter of lower portion 247 approximating the inside clear diameter of spacer ring 200. Lower portion 247 is centrally mounted on the underside of upper portion 239 such that a circumferential overhang 253 is created by upper portion 239 extending outward past the periphery of lower portion 247. Overhang 253 may include a notch 256 for receiving the top of spacer ring 200.

Plant feature 217 is placed on spacer ring 200 which is positioned on warming receptacle 20. The lower portion 247 of plant feature 217 is inserted into the clear space of the spacer ring and overhang 253 rests on top of spacer ring 200. If overhang 253 has a notch 256, the top of spacer ring 200 fits in the notch.

Plant accent 237 includes a plant 262. As shown in the drawings, the present embodiment comprises a plant 262 comprising synthetic flowers 264; however as noted above the feature may have other types of plant themed objects (both synthetic and organic). The flowers 264 include stems connected to a central trunk 267. As shown in FIG. 5, trunk 267 passes through mount 234 and is held therein by a retaining member 270 lower portion 247. Retaining member 270 may be any device commonly known in the art such as staples, adhesive, tacks or any other method of retaining synthetic floral stems in a piece of material. It is foreseen that mount 234 could be made of foam or other type of easily penetrated material and a retaining member 270 may not be necessary because the trunk 267 can be pushed into position without completely penetrating mount 234.

Scent dispersing apparatus 10 is used by placing warming receptacle 20 inside upper opening 48 of housing 12 such that it rests on ledge 51. Next, blocks of scent producing substance 84 are added to the warming receptacle 20. Plant feature 17 is then placed atop warming receptacle 20, or if a spacer ring 200 is used, plant feature 217 is placed atop the spacer ring 200 which is atop the warming receptacle 20. Finally, the electric heat source 23 is activated, which melts the scent producing substance 84 to release the scent. The scent flows through vents 64 and 100, to the area surrounding apparatus 10. Thus, a user can enjoy the scent as well as the aesthetics of the plant feature.

It should be understood that while certain forms and embodiments have been illustrated and described herein, the present invention is not to be limited to the specific forms or arrangement of parts described and shown, and that the various features described may be combined in ways other than those specifically described without departing from the scope of the present invention. The terms “substantially,” “generally,” “approximately,” or any other qualifying term as used herein may be applied to modify any quantitative representation, which could permissibly vary without resulting in a change to the basic function to which it is related.

It is to be understood that while certain forms of this invention have been illustrated and described, it is not limited thereto, except in so far as such limitations are included in the following claims and allowable equivalents thereof.

Having thus described the invention, what is claimed as new and desired to be secured by Letters Patent is:

1. A scent dispersing apparatus comprising:
a shell extending upwardly from a base, said shell defining an interior void;
a ledge extending from a wall of said shell into said interior void;
a receptacle for holding a scent producing substance, a portion of said receptacle positioned atop said ledge, said receptacle having a sidewall and a ventilation opening formed in said sidewall; and
a plant feature, said plant feature comprising a synthetic or organic plant extending upwardly from a mount, said mount positioned on said receptacle.

2. The scent dispersing apparatus of claim 1, further comprising an electric heat source.

3. The scent dispersing apparatus of claim 2, wherein said electric heat source is secured to said base below said receptacle, said electric heat source adapted to melt said scent producing substance.

4. The scent dispersing apparatus of claim 3, wherein said electric heat source gives off light.

5. The scent dispersing apparatus of claim 3, wherein said shell has a ventilation opening formed in said wall of said shell.

6. The scent dispersing apparatus of claim 5, wherein said ventilation opening formed in said wall of said shell is located below said ledge.

7. The scent dispersing apparatus of claim 6, wherein said mount has a channel into which a portion of said receptacle is inserted.

8. A scent dispersing apparatus comprising:
a receptacle holding a scent producing substance, said receptacle having a sidewall and a ventilation opening formed in said sidewall;
a shell extending upwardly from a base, said shell supporting said receptacle;
a plant feature, said plant feature comprising a synthetic or organic plant; and
a spacer positioned between said receptacle and said plant feature.

9. The scent dispersing apparatus of claim 8, further comprising an electric heat source.

10. The scent dispersing apparatus of claim 9, wherein said electric heat source is secured to said base below said receptacle, said electric heat source adapted to melt said scent producing substance.

11. The scent dispersing apparatus of claim 10, wherein said electric heat source gives off light.

12. The scent dispersing apparatus of claim 10, wherein said shell has a ventilation opening formed in a wall of said shell.

13. The scent dispersing apparatus of claim 12, wherein said spacer has a channel into which a portion of said receptacle is inserted.

14. A scent dispersing apparatus comprising:
a shell extending upwardly from a base and defining an interior void, said shell having a ventilation opening formed in a wall of said shell;
a ledge extending from said wall of said shell into said interior void;
a receptacle for holding a scent producing substance, a portion of said receptacle positioned atop said ledge, said receptacle having a sidewall and a ventilation opening formed in said sidewall;
a plant feature supported by said receptacle; and
an electric heat source located in said interior void below said receptacle, said electric heat source adapted to melt said scent producing substance.

15. The scent dispersing apparatus of claim 14, wherein said electric heat source gives off light.

16. The scent dispersing apparatus of claim 15, wherein said ventilation opening formed in said wall of said shell is located below said ledge.

17. The scent dispersing apparatus of claim 16, wherein said plant feature comprises a synthetic or organic plant extending upwardly from a mount.

18. The scent dispersing apparatus of claim 17, wherein said synthetic or organic plant is attached to said mount in more than one location on said mount.

19. The scent dispersing apparatus of claim 18, wherein said scent producing substance gives off a scent that matches a scent that said synthetic or organic plant would give off naturally if said synthetic or organic plant was found in nature.

20. The scent dispersing apparatus of claim 19, wherein said shell is cylindrical.
